SEQUENCE LISTING (1 sequence)

SEQ ID NO: 1 - protein, 163 aa, Homo sapiens
MADEEKLPPGWEKRMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

(19) United States
(12) Patent Application Publication
(10) Pub. No.: US 2004/0176277 A1
(43) Pub. Date: Sep. 9, 2004

(54) Methods of treating Pin1 associated disorders

(75) Inventors: Janusz M. Sowadski, Boston, MA (US); Robert K. Suto, Maynard, MA (US)

Correspondence Address: LAHIVE & COCKFIELD, LLP., 28 STATE STREET, BOSTON, MA, 02109, US

(73) Assignee: Pintex Pharmaceuticals, Inc., Watertown, MA (US)

(21) Appl. No.: 10/379,161

(22) Filed: Mar. 3, 2003

(51) Int. Cl. A61K 38/16; A61K 31/66

(52) U.S. Cl. 514/7; 514/114

(57) ABSTRACT

This invention pertains to methods of treating Pin1 associated disorders through the administration of inhibitors of the Pin1 isomerase activity.

This application claims priority to U.S. Provisional Application No. 60/360,799 filed Mar. 1, 2002.

### RELATED APPLICATIONS

This application is related to U.S. application Ser. No. 60/257,412, filed Dec. 22, 2000; U.S. application Ser. No. 09/726,464, filed Nov. 29, 2000; U.S. application Ser. No. 08/988,842, filed Dec. 11, 1997; and WO 99/12962, published Mar. 8, 1999, the entire contents of each of which are expressly incorporated herein by reference. PTZ-034, entitled “Pin1-Modulating Compounds and Methods of Use Thereof”, filed Mar. 3, 2003; PTZ-035, entitled “Pin1-Modulating Compounds and Methods of Use Thereof”, filed Mar. 3, 2003; PTZ-036, entitled “Pin1-Modulating Compounds and Methods of Use Thereof”, filed Mar. 3, 2003; U.S. Provisional Application Serial No. 60/361,246, filed Mar. 1, 2002; U.S. Provisional Application Serial No. 60/361,206, filed Mar. 1, 2002; and U.S. Provisional Application Ser. No: 60/361,231, filed Mar. 1, 2002 the entire contents of which are expressly incorporated by reference.

### TECHNICAL FIELD OF THE INVENTION

This invention relates to methods for treating disorders associated with the peptidyl prolyl isomerase, Pin1. The methods focus on treating these disorders with modulators, e.g., inhibitors, of the isomerase activity of Pin1.

### BACKGROUND OF THE INVENTION

The peptidyl-prolyl cis-trans isomerases (PPIases), or rotamases, are a family of ubiquitous enzymes that catalyze the cis/trans isomerization of the peptide bond on the n-terminal side of proline residues in proteins (Hunter, Cell 92:141-142, 1998). PPIases are divided into three classes, cyclophilins (Cyps), FK-506 binding proteins (FKBPs) and the Pin1/parvulin class.

Cyclophilins and FKBPs are distinguished by their ability to bind the clinically immunosuppressive drugs cyclosporin and FK506, respectively (Schreiber, Science 251:283-7, 1991; Hunter, supra). Upon binding of these drugs, there are two common outcomes: inhibition of the PPIase activity and inhibition of the common target calcineurin. The inhibition of calcineurin phosphatase activity prevents lymphocytes from responding to antigen-induced mitogenic signals, thus resulting in immunosuppression. However, the inhibition of the PPIase activity is apparently unrelated to the immunosuppressive property of the drug/PPIase complexes. Even more surprisingly, deletion of all 8 known cyclophilins and 4 FKBPs in the same cells does not result in any significant phenotype (Dolinski et al., Proc. Natl. Acad. Sci. USA 94:13093-131098, 1997).

In contrast, members of the Pin1/ parvulin class of PPIases bind neither of these immunosuppressive drugs, and are structurally unrelated to the other two classes of PPIases. Known members of the Pin1/parvulin class include Pins1-3 (Lu et al., Nature 380;544-547, 1996), Pin-L (Campbell et al., Genomics 44:157-162, 1997), parvulin (Rahfeld, et al., Proc. Natl. Acad. Sci. USA 93:447-451, 1996) and Ess1/Pft1 (Hanes et al., Yeast 5:55-72, 1989; and Hani, et al. FEBS Letts 365:198-202, 1995).

Pin1 is a highly conserved protein that catalyzes the isomerization of only phosphorylated Ser/Thr-Pro bonds (Rananathan, R. et al. (1997) Cell 89:875-86; Yaffe, et al. 1997, Science 278:1957-1960; Shen, et al. 1998,Genes Dev. 12:706-720; Lu, et al. 1999, Science 283:1325-1328; Crenshaw, et al. 1998, Embo J. 17:1315-1327; Lu, et al. 1999, Nature 399:784-788; Zhou, et al. 1999, Cell Mol. Life Sci. 56:788-806). In addition, Pin1 contains an N-terminal WW domain, which functions as a phosphorylated Ser/Thre-Pro binding module (Sudol, M. (1996) Prog. Biophys. Mol. Biol. 65:113-32). This phosphorylation-dependent interaction targets Pin1 to a subset of phosphorylated substrates, including Cdc25, Wee 1, Mytl, Tau-Rad4, and the C-terminal domain of RNA polymerase II large domain (Crenshaw, D. G., et al. (1998) Embo. J. 17:1315-27; Shen, M. (1998) Genes Dev. 12:706-20; Wells, N. J. (1999) J Cell. Sci. 112:3861-71).

The specificity of Pin1 activity is essential for cell growth; depletion or mutations of Pin1 cause growth arrest, affect cell cycle checkpoints and induce premature mitotic entry, mitotic arrest and apoptosis in human tumor cells, yeast or Xenopus extracts (Lu, et al. 1996, Nature 380:544-547; Winkler, et al. 200, Science 287:1644-1647; Hani, et al. 1999. J. Biol. Chem. 274:108-116). In addition, Pin1 is dramatically overexpressed in human cancer samples and the levels of Pin1 are correlated with the aggressiveness of tumors. Moreover, inhibition of Pin1 by various approaches, including Pin1 antisense polynucleotides or genetic depletion, kills human and yeast dividing cells by inducing premature mitotic entry and apoptosis.

Thus, Pin1-catalyzed prolyl isomerization regulates the conformation and function of these phosphoprotein substrates and facilitates dephosphorylation because of the conformational specificity of some phosphatases. Thus, Pin1-dependent peptide bond isomerization is a critical post-phosphorylation regulatory mechanism, allowing cells to turn phosphoprotein function on or off with high efficiency and specificity during temporally regulated events, including the cell cycle (Lu et al., supra).

Taken together, these results indicate that the Pin-1 subfamily of enzymes is a novel target for diseases characterized by uncontrolled cell proliferation, primarily malignancies. Therefore, there is an ongoing need for specific inhibitors of Pin1 and Pin1-related proteins, and for reliable methods of designing such inhibitors.

### SUMMARY OF THE INVENTION

The present invention provides a method for treating a subject suffering from a Pin1 associated disorder.

In one embodiment the invention pertains to a method of treating a Pin1 associated disorder by administering an inhibitor of Pin1 activity having any one or more of the following moieties:

A-B-C-D-E

where A is a hydrophobic pocket interacting moiety; where B is a cysteine/serine valley interacting moiety; where C is a phosphate pocket interacting moiety; and where D is a substrate entry groove interacting moiety and where E is a lip region interacting moiety such that administration of any combination of these moieties treats the Pin1 disorder.

In related embodiments, the inhibitor of the invention can contain any one, two, three, four or five of the interacting moieties and these moieties can be covalently linked.

### BRIEF DESCRIPTION OF THE DRAWINGS

FIG. 1 depicts the amino acid sequence of Pin1 (SEQ ID NO:1).

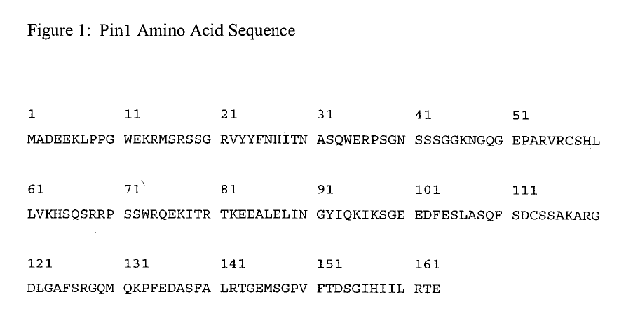

FIG. 2 is a schematic representation of the defined areas of the peptidyl polyl isomerase active site of Pin1.

### DETAILED DESCRIPTION

Definitions

The term “interacting agent” includes substances which can interact with the Pin1 polypeptide such that the three-dimensional structure can be determined. In one embodiment, the interacting agent is a Pin1 inhibitor, a substrate-derived peptide, or a solvent based molecule.

The term “co-complex” refers to a Pin1 polypeptide, Pin1 related polypeptide or fragment thereof in covalent or non-covalent association with a chemical entity or compound.

The term “modulator of Pin1” are compounds that have the ability to modulate the activity of Pin1 (SEQ ID NO.: 1). These modulators can be, for example, Pin1 inhibitors.

The term “Pin1 inhibitor” refers to any molecule that can interact with Pin1 or a Pin1-related polypeptide and inhibit the ability of the polypeptide to carry out proline isomerization activity. Compounds within the scope of the invention can be naturally occurring or chemically synthesized. The term is also intended to include pharmaceutically acceptable salts of the compounds. In certain embodiments, the inhibitor is specific for Pin1, i.e., does not inhibit the isomerase activity of PPIases belonging to other classes (e.g., cyclophilins or FKBPs). In at least one embodiment, the Pin1 inhibitor is not Fredericamycin A or a Fredericamycin-related compound.

The term “Pin1-associated state” or “Pin1 associated disorder” includes disorders and states (e.g., a disease state) which are associated with abnormal cell growth, abnormal cell proliferation, or aberrant levels of Pin1 marker. Pin1-associated states include states resulting from an elevation in the expression of cyclin D1 and/or Pin1. Pin1-associated states also include states resulting from an elevation in the phosphorylation level of c-Jun, particularly phosphorylation of c-Jun on Ser^{63/73}-Pro and/or from an elevation in the level of c-Jun amino terminal kinases (JNKs) present in a cell. Pin1-associated states include neoplasia, cancer, undesirable cell growth, and/or tumor growth. Pin1-associated states include states caused by DNA damage, an oncogenic protein (i.e. Ha-Ras), loss of or reduced expression of a tumor suppressor (i.e. Brca1), and/or growth factors.

The language “Pin1 inhibited-state” is intended to include states in which one activity of Pin1 is inhibited in cells, e.g., cells in a subject, that have been treated with a Pin1 modulating compound. “Pin1 inhibited-state” is also intended to include states wherein the Pin1 modulating compound is administered to a subject, allowed to remain in a preactivated state, and subsequently activated by a stimulus. The stimulus may be selected from a natural event, artificial event, or the combination thereof. For example, the natural event may be the action of an enzyme and/or the artificial event may be the addition of a hyperplastic inhibitory agent or the addition of energy to the subjects system in any manner that achieves activation, e.g., by radiation, e.g., by light with a wavelength greater than about 400 nm, e.g., greater than about 600 nm, e.g., greater than about 620 nm, e.g., greater than about 630 nm, e.g., greater than about 640 nm, e.g., greater than about 650 nm. In one embodiment, the cells enter a Pin1 inhibited-state for a designated period of time prior to activation of the modulating compound sufficient to allow the modulation the activity of Pin1 by the activated modulating compound. In certain embodiments of the invention, the designated period of time prior to activation is greater than about 1 hour, e.g., greater than about 2 hours, e.g., greater than about 3 hours, e.g., greater than about 6 hours, e.g., greater than about 12 hours, e.g., greater than about 24 hours, e.g., greater than about 36 hours, e.g., greater than about 48 hours, e.g., greater than about 72 hours. In a specific embodiment, the designated period of time prior to activation is 3 days. In one embodiment, the Pin1 modulating compound is preactivated prior to administration to a subject followed by the introduction of at least one stimulus sufficient to allow the modulation the activity of Pin1 by the modulating compound. In certain embodiment of the invention, the activity of the modulating compound is enhanced by the entrance of the cells, e.g., cells of a subject, into a Pin1 inhibited state.

A “competitive” inhibitor as used herein is one that inhibits proline isomerase activity by binding to the same kinetic form of the enzyme as its substrate binds, thus directly competing with the substrate for the active site. Competitive inhibition can be reversed completely by increasing the substrate concentration.

An “uncompetitive” inhibitor as used herein is one that inhibits proline isomerase activity by binding to a different kinetic form of the enzyme than does the substrate. Such inhibitors bind to the enzyme already bound with the substrate and not to the free enzyme. Uncompetitive inhibition cannot be completely reversed by increasing the substrate concentration.

A “noncompetitive” inhibitor as used herein is one that can bind to either the free or substrate bound enzyme.

The term “interaction template” refers to a three dimensional model built using information obtained from crystal structures solved with chemical entities bound in the active site. The interaction template is formed by using a Pin1 polypeptide having a substitution insertion or deletion of one or more amino acids of the amino acid sequence set forth in SEQ ID NO:1, wherein the active site of the Pin1 polypeptide is accessible to solvent and available for interaction with modulators, e.g., inhibitors. This crystallized form of Pin1 is used to facilitate solving high resolution molecular structures with molecules bound in the active site such that the residues capable of interacting with inhibitors in the active site are determined. This template is used in the design of high affinity inhibitors of Pin1 isomerase activity.

The term “specificity template” refers to a template created by comparing sequence alignments of homologous or functionally related proteins. Identification of conserved and non-conserved residues allows a skilled artisan to design inhibitors of prolyl isomerase activity that increase affinity and specificity. The determination of conserved amino acids allows the skilled artisan to develop inhibitors with increased affinity but not, necessarily, specificity. The determination of non conserved amino acids allows the skilled artisan to develop inhibitors that have increased affinity and specificity.

The term “hydrophobic pocket” refers to the portion of the active site that binds a hydrophobic moiety. In one embodiment, the hydrophobic pocket contains 4, 6, 8, 10, 12 or 14 hydrophobic amino acid residues. In one particular embodiment, the hydrophobic pocket contains amino acid residues His59, Leu61, Leu122, Phe125, Met130, Gln131, Pro133, Phe134, Thr152, and His157 of SEQ ID NO:1.

The term “cysteine/serine valley” refers to a portion of the active site that is responsible for binding the serine moiety of the substrate. In one embodiment this region contains residues Leu61, Cys113 and Ser154 of SEQ ID NO:1.

The term “phosphate binding pocket” refers to a region of the active site containing three positively charged amino acids that binds negatively charged moieties or hydrogen donor/acceptor groups. In one embodiment, this pocket is contains 4, 6, 8, or 10 amino acid residues. In one particular embodiment, this pocket is defined by residues Lys63, Ser67, Arg68, Arg69, Pro70 and Ser154.

The term “substrate entry groove” refers to a region of the polypeptide that allows for substrate entry into the active site. In one embodiment this groove contains amino acids Lys63, Arg69, Ser71, Ser72, Trp73, Arg74, Gln75, Glu76, Asp112, Cys113, Ser114.

The term “lip regions” refers to the residues that surround the active site cavity, as defined previously. In one embodiment these lip regions contain residues that are within 10 Å of the active site cavity. In one particular embodiment, this lip region is defined by, but not limited to, residues Arg54, Arg56, His64, Ser65, Gln66, Lys77, Ile78, Thr79, Ser115, Lys117, Ala118, Gly123, Ala124, Phe125, Ser126, Arg127, Gly128, Gln129, Pro133, Glu135, Lys132, Phe151, Asp153, Gly155, and Ile156.

The term “hydrophobic pocket interacting moiety” refers to a compound that is capable of interacting with the residues of the hydrophobic pocket. This interaction can be, for example, through hydrophobic interactions, through van der Waals contacts, or through hydrogen bonds.

The term “cysteine/serine valley interacting moiety” refers to a compound that is capable of interacting with Cys113 or Ser154, i.e., the cysteine/serine valley, within the region of the active site that binds the serine residue of the natural substrate. This interaction can be covalent, noncovalent, through hydrogen bonds, or by van der Waals interactions.

The term “phosphate binding pocket interacting moiety” refers to a compound that can interact with the phosphate binding pocket. This interaction can be electrostatic, through salt bridges, covalent, or through van der Waals interactions.

The term “substrate entry groove interacting moiety” refers to a compound that can interact with the substrate entry groove. This interaction can be electrostatic, hydrophobic, covalent, hydrogen bonding, or through van der Waals interactions

The term “lip region interacting moiety” refers to a compound that is capable of interacting with the residues outside the active site. This interaction can be, for example through hydrophobic interactions, though van der Waals contacts, or through hydrogen bonds.

The term “radiation therapy” includes the application of a genetically and somatically safe level of electrons, protons, or photons, both localized and non-localized, to a subject to inhibit, reduce, or prevent symptoms or conditions associated with undesirable cell growth. The term X-rays is also intended to include machine-generated radiation, clinically acceptable radioactive elements, and isotopes thereof, as well as the radioactive emissions therefrom. Examples of the types of emissions include alpha rays, beta rays including hard betas, high-energy electrons, and gamma rays. Radiation therapy is well known in the art (see e.g., Fishbach, F., Laboratory Diagnostic Tests, 3rd Ed., Ch. 10: 581-644 (1988)), and is typically used to treat neoplastic diseases.

I. Architecture of the Pin1 Active Site

In copending application (U.S. Ser. No. 10/_______), entitled, “Methods for Designing Specific Inhibitors for Pin1 Proline Isomerase and Pin1 Related Molecules,” filed on even date herewith, and expressly incorporated by reference, a method of defining the interaction template and specificity template of Pin1 is disclosed. Through the use of X-ray crystallography and co-crystals of Pin1 polypeptides with molecules bound in the active site, the regions of the active site of Pin1 are defined. Five regions of the active are shown to exist. The five regions are: the phosphate binding pocket; the cysteine/serine valley; the hydrophobic pocket; the substrate entry groove; and lip region. The entire copending application is expressly incorporated by reference herein and the description pertaining to the Pin1 active site is reiterated herein.

II. Methods of Treating Pin1-Associated Disorders

In one aspect, the invention includes methods of treatment using inhibitors of PPlase activity. Enzymes of the Pin1/parvulin class of PPIases are known to be essential for mitosis. Such enzymes have been identified in bacteria, fungi, insect and mammalian cells. Thus the compounds of the invention are useful for the treatment of a wide variety of disorders involving mitosis and cell proliferation.

A. Pin-Associated States

Accordingly, the term “Pin1-associated state” as used herein includes a disorder or a state (e.g., a disease state) that is associated with abnormal cell growth, abnormal cell proliferation, or aberrant levels of Pin1 marker. Pin1-associated states include states resulting from an elevation of cyclin D1 and/or Pin 1. Pin1-associated state also includes states resulting from an elevation in the phosphorylation level of c-Jun, particularly phosphorylation of c-Jun on S^{63/73}-P and/or from an elevation in the level of c-Jun amino terminal kinases (JNKs) present in a cell. Pin1-associated states further include states caused by DNA damage, an oncogenic protein (i.e., Ha-Ras), loss of or reduced expression of a tumor suppressor (i.e., Brca1), and/or growth factors.

As used herein, the term “abnormal cell growth” is intended to include cell growth which is undesirable or inappropriate. Abnormal cell growth also includes proliferation which is undesirable or inappropriate (e.g., unregulated cell proliferation or undesirably rapid cell proliferation). Abnormal cell growth can be benign and result in benign masses of tissue or cells, or benign tumors. Many art-recognized conditions are associated with such benign masses or benign tumors including diabetic retinopathy, retrolental fibrioplasia, neovascular glaucoma, psoriasis, angiofibromas, rheumatoid arthritis, hmangiomas, and Karposi's sarcoma. Abnormal cell growth can also be malignant and result in malignancies, malignant masses of tissue or cells, or malignant tumors. Many art-recognized conditions and disorders are associated with malignancies, malignant masses, and malignant tumors.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to grow and function somewhat independently of the homeostatic mechanisms which control normal tissue growth and function. However, some neoplasms remain under the control of the homeostatic mechanisms which control normal tissue growth and function. For example, some neoplasms are estrogen sensitive and can be arrested by anti-estrogen therapy. Neoplasms can range in size from less than 1 cm to over 6 inches in diameter. A neoplasm even 1 cm in diameter can cause biliary obstructions and jaundice if it arises in and obstructs the ampulla of Vater.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histologic and others features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tend to grow more slowly than malignant tumors. Benign neoplasms also tend to be less autonomous than malignant tumors. Benign neoplasms tend to closely histologically resemble the tissue from which they originated. More highly differentiated cancers, cancers that resemble the tissue from which they originated, tend to have a better prognosis than poorly differentiated cancers. Malignant tumors are more likely than benign tumors to have aberrant functions (i.e. the secretion of abnormal or excessive quantities of hormones).

The histological features of cancer are summarized by the term “anaplasia.” Malignant neoplasms often contain numerous mitotic cells. These cells are typically abnormal. Such mitotic aberrations account for some of the karyotypic abnonnalities found in most cancers. Bizarre multinucleated cells are also seen in some cancers, especially those which are highly anaplastic. “Dyplasia” refers to a pre-malignant state in which a tissue demonstrates histologic and cytologic features intermediate between normal and anaplastic. Dysplasia is often reversible. “Anaplasia” refers to the histological features of cancer. These features include derangement of the normal tissue architecture, the crowding of cells, lack of cellular orientation termed dyspolarity, cellular heterogeneity in size and shape termed “pleomorphism.” The cytologic features of anaplasia include an increased nuclear-cytoplasmic ratio (nuclear-cytoplasmic ratio can be over 50% for malignant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decrease in size and number of organelles, enlarged and increased numbers of nucleoli, and sometimes the presence of intermediate filaments.

As used herein, the term “cancer” includes a malignancy characterized by deregulated or uncontrolled cell growth, for instance carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. An “adenocarcinoma” refers to a carcinoma derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the therapeutic methods of the present invention can be used to treat many kinds of cancers (e.g., oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kidney chromophobic carcinoma, kidney clear cell carcinoma, kidney oncocytoma, kidney papillary carcinoma, testis non-seminomatous cancer, testis seiminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, liposarcoma abnormal cell growth. Specifically, Pin1 has been shown to be overexpressed in the tumor types disclosed in Table 1.

The language “inhibiting undesirable cell growth” is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by dysregulated endothelial cell division.

“Inhibiting tumor growth” or “inhibiting neoplasia” is intended to include the prevention of the growth of a tumor in a subject or a reduction in the growth of a pre-existing tumor in a subject. The inhibition also can be the inhibition of the metastasis of a tumor from one site to another. In particular, the language “tumor” is intended to encompass both in vitro and in vivo tumors that form in any organ or body part of the subject. The tumors preferably are tumors sensitive to the fredericamycin A compounds of the present invention. Examples of the types of tumors intended to be encompassed by the present invention include those tumors associated with breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidneys. Specifically, the tumors whose growth rate is inhibited by the present invention include basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforna, leukemias, lymphomas (i.e. maglinant lymphomas, mantle cell lymphoma), malignant melanomas, multiple myeloma, epidermoid carcinomas, and other carcinomas and sarcomas.

The language “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharniacological Basis of Therapeutics, 8th Ed., Sec 12:1202-1263 (1990)), and are typically used to treat neoplastic diseases. The chemotherapeutic agents generally employed in chemotherapy treatments are listed below in Table 3. Other similar examples of chemotherapeutic agents include: bleomycin, docetaxel (Taxotere), doxorubicin, edatrexate, etoposide, finasteride (Proscar), flutamide (Eulexin), gemcitabine (Gemzar), goserelin acetate (Zoladex), granisetron (Kytril), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

Additional cell proliferative disorders contemplated for treatment using the compounds and methods of the invention include fibrotic disorders, non-neoplastic growths such as benign prostatic hypertrophy, endometriosis, psoriasis, and the like.

Fibrotic disorders are generally characterized by inappropriate overproliferation of non-cancerous fibroblasts. Examples include fibromyalgia, fibrosis (systic, hepatic, idopathic pulmonary, pericardial, and the like), cardiac fibromas, fibromuscular hyperplasia, restenosis, athersclerosis, fibromyositis, and the like.

The compounds of the invention are additionally useful in inhibiting mitosis in pathogenic organisms and are therefore useful for treating infectious diseases. Particular infectious diseases treatable by the methods disclosed herein include bacterial infections and fungal infections.

Bacterial infections contemplated for treatement using the compounds of the invention include infections caused by both gram-positive and gram-negative bacteria, including infections caused by Staphylococcus, Clostridium, Streptococcus, Enterococcus, Diplococcus, Hemophilus, Neisseria, Erysipelothricosis, Listeria, Bacillus, Salmonells, Shigella, Escherichia, Klebsiellla, Enterobacter, Serratia, Proteus, morganells, Providencia, Yersinia, Camphylobacter, Myobacteria, and the like. Infection by such organisms causes a wide variety of disorders including pneumonia, diarrhea and dysentery, anthrax, rheumatic fever, toxic shock syndrome, mastoiditis, menigitis, gonorrhea, typhoid fever, gastoeneritis, brucellosis, cholera, bubonic plague, tetanus, tuberculosis, Lyme disease, and the like.

Fungal infections contemplated for treatment using the compounds of the invention include fungal infections , dermatophytoses and fungal infections of the genito-urinary tract. Systemic fungal infections include those caused by Histoplasma, Coccidiodes, Cryptococcus, Blastocyces, Paracoccidioides, Candida, Asperfillus, Nocardia, Sporothrix, Rhizopus, Absidia, Mucor, Hormodendrum, Phialophora, Rhinosporidium, and the like. Dermatophyte infections include those caused by Microsporum, Trichophyton, Epidermophyton, Candida, Pityrosporum, and the like. Fungal disorders of the genito-urinary tract include infections caused by Candida, Cryptococcus, Aspergillus, Zygomycodoides, and the like. Infection by such organisms causes a wide variety of disorders such as ringworm, thrush, San Joaquin fever or Valley fever, Gilcrist's disease, and the like. These infections can be particularly serious, and even fatal, in patients with a depressed immune system such as organ transplant recipients and persons with acquired immunodeficiency syndrome (AIDS).

B. Administration of the Pin1-Inhibitors

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. Most preferably the subject is a human.

The language “effective amount” of the compound is that amount necessary or sufficient to treat or prevent a Pin1 associated state, e.g. prevent the various morphological and somatic symptoms of a Pini associated state. In an example, an effective amount of a Pin1 inhibitor of the invention is the amount sufficient to inhibit undesirable cell growth in a subject. In another example, an effective amount of the Pin1 inhibitor compound is the amount sufficient to reduce the size of a pre-existing benign cell mass or malignant tumor in a subject. The effective amount can vary depending on such factors as the size and weight of the subject, the type of illness, or the particular Pin1 binding compound. For example, the choice of the Pin1 inhibitor compound can affect what constitutes an “effective amount”. One of ordinary skill in the art would be able to study the aforementioned factors and make the determination regarding the effective amount of the Pin1 binding compound without undue experimentation. In one possible assay, an effective amount of a Pin1 inhibitor compound can be determined by assaying for the expression of cyclin D1 and determining the amount of the Pin1 inhibitor compound sufficient to reduce the levels of cyclin D1 to that associated with a non-cancerous state.

The regimen of administration can affect what constitutes an effective amount. The Pin1 inhibitor compound can be administered to the subject either prior to or after the onset of a Pin1 associated state. Further, several divided dosages, as well as staggered dosages, can be administered daily or sequentially, or the dose can be continuously infused, or can be a bolus injection. Further, the dosages of the Pin1 inhibitor(s) can be proportionally increased or decreased as indicated by the exigencies of the therapeutic or prophylactic situation.

The term “treated,” “treating” or “treatment” includes the diminishment or alleviation of at least one symptom associated or caused by the state, disorder or disease being treated. For example, treatment can be diminishment of one or several symptoms of a disorder or complete eradication of a disorder.

While it is possible for a compound of the present invention to be administered alone, it is preferable to administer the compound as a pharmaceutical composition.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agaragar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an Pin1 associated state. If desired, the effective daily dose of the active compound may be administered as two, three, four, five, six or more sub-doses administered separately at appropriate intervals throughout the day, optionally, in unit dosage forms.

III. Inhibitors of Pin1

The crystallized Pin1 R14 A and Pin1 R14A-inhibitor complexes have, for the first time, provided structural information that has enabled identification of the regions of the Pin1 polypeptide involved in the molecular interaction with Pin1 inhibitors, thereby providing methods for identifying specific inhibitors of Pin1. By comparison of the crystal structures of Pin1 R14A and the Pin1 R14A-inhibitor complexes described herein, potential sites for modification within the inhibitors have been identified providing an important tool for determining the most efficient binding interactions between Pin1 and a potential inhibitor.

The Pin1 polypeptide consists of two structural domains organized around a hydrophobic cavity. The N-terminal WW domain, defined as residues 1-39 and a C-terminal proline isomerase domain, defined as residues 45-163. The WW domain is a three-stranded anti-parallel β-sheet. The C-terminal proline isomerase domain is a 4 stranded anti-parallel β-sheet surrounded by 4 α helices (e.g., Ranganathan, et al Cell 89:875-886, 1997; Verdecia et al., Nat Struct Biol 7(8):639-43, 2000).

Based on the comparison of the structural coordinates of the crystals of Pin1 R14A and co-complexes, regions of the active site involved in key interactions which define Pin1 enzyme activity and specificity were elucidated. As shown in FIG. 6, these regions include the following:

The “hydrophobic pocket” is composed of residues His59, Leu61, Leul22, Phe125, Met130, Gln131, Pro133, Phe134, Thr152, His157 of Pin1 (SEQ ID NO.1). This pocket binds the hydrophobic side chain of the substrate proline.

Residues Leu61, Cys113, and Ser154 in the active site are here defined as a “cysteine/serine valley”, residing between the “hydrophobic pocket” and the “phosphate binding pocket”. The isomerized peptide bond of an alanine-proline dipeptide was found to be located in this region.

The third region is the “phosphate binding pocket” consisting of a region of high localized positive charge and is defined with amino acids Lys63, Ser67, Arg68, Arg69, Pro70, and Ser154. This region is part of the specificity loop (amino acids 64-80) for the phosphate-specific recognition by Pin1 of phosphorylated serine/threonine peptide substrates which are uniquely recognized and isomerized the Pin1 family of proline isomerases, in contrast to FK506 binding proteins and cyclophilins.

A fourth region is the “substrate entry groove” as defined by the following amino acids: Lys63, Arg69, Ser71, Ser72, Trp73, Arg74, Gln75, Glu76, Asp112, Cys113, Ser114.

A fifth region is the “lip regions” as defined by the residues that surround the active site cavity. In one embodiment these lip regions contain residues that are within 10 Å of the active site cavity. In one particular embodiment, this lip region is defined by, but not limited to, residues Arg54, Arg56, His64, Ser65, Gln66, Lys77, Ile78, Thr79, Ser 115, Lys 117, Ala 118, Gly123, Ala124, Phe125, Ser126, Arg127, Gly128, Gln129, Pro133, Glu135, Lys132, Phe151, Asp153, Gly155, and Ile156.

Accordingly, in another aspect, the invention provides a method of designing specific inhibitors of Pin1 isomerase activity based on the ability of molecules to bind to one or more of the defined regions of the Pin1 proline isomerase active site.

In one embodiment, the method uses the structure coordinates of Pin1 R14A and/or Pin1 R14A-inhibitor complexes to design compounds that bind to at least one of the regions of the active site. In certain embodiments, the compounds are designed to bind at least one, preferably to at least two, more preferably at least three, and most preferably all four of the regions of the active site.

In another embodiment, the method uses a crystal of Pin1R14A probed with molecules composed of a variety of different chemical moieties to determine optimal sites for interaction between candidate Pin1 inhibitors and the enzyme. For example, small molecules that bind tightly to one or more of the four regions of the active site can be designed and tested for their ability to inhibit Pin1 isomerase activity using the assays described herein.

Another method included in this aspect of the invention is to screen computationally small molecule databases for chemical entities that can bind in whole, or in part to, to one or more of the regions of the Pin1 active site. In one embodiment of this method, the quality or fit of the compound identified to the regions of the Pin1 active site can be judged either by shape complementarity or by estimated interaction energy (Meng et al., J. Comp. Chem. 13:505-524, 1992).

In a further embodiment, potential inhibitors that can be analyzed according to the methods of the invention can be obtained using any of the numerous approaches in combinatorial library methods known in the art. In this embodiment, potential inhibitors are first identified for Pin1 inhibitory activity using the in vitro assays described herein. Once potential inhibitors are identified, and their structures determined, further optimization can be carried out by computational analyses using the structure coordinates of the Pin1 R14A and Pin1R14A-co-complexes described herein. Alternatively, further optimization can be carried out by determining the structural coordinates of crystallized co-complexes of the potential inhibitor and Pin1 R14A using the methods described herein.

Various combinatorial libraries that can be used in the methods of the invention include, but are not limited to: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Further, examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J Mol. Biol. 222:301-310); (Ladner supra.).

The potential inhibitory effect of a compound can be further analyzed prior to its actual synthesis and testing by use of computer modeling techniques using the structural coordinates of the Pin1 R14A and Pin1 R14A-inhibitor co-complexes described herein. If the computer modeling indicates a strong interaction, the molecule can then be synthesized using standard methods known to those skilled in the chemical arts, and then tested for its ability to inhibit Pin1 isomerase activity using the assays set forth herein.

An inhibitory or other binding compound of Pin1 may be computationally evaluated and designed by means of a series of steps in which chemical entities or fragments are screened and selected for their ability to associate with the individual binding pockets or other areas of Pin1.

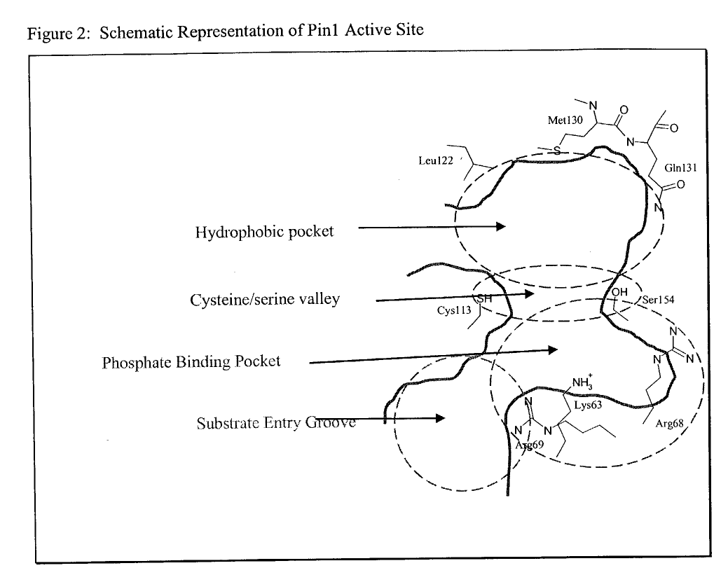

In other embodiments of the method of the invention, potential inhibitory compounds can be examined for their ability to associate with Pin1 and more particularly with the four regions of the Pin1 active site. This process can involve visual inspection of, for example, the active site on the computer screen based on the coordinates of the Pin1 R14A and Pin1 R14A-inhibitor complex as set forth in FIGS. 2-4. Selected compounds or chemical moieties can then be positioned in a variety of orientations, or docked, within an individual region of the Pin1 active site as defined herein. Docking can be accomplished using software such as Quanta and SYBYL, followed by energy minimization and molecular dynamics with standard molecular mechanics forcefields, such as CHARMM and AMBER.

Specialized computer programs that can also be used in the process of selecting compounds or chemical entities include:

1. SYBYL Available from Tripos Inc., 1699 South Hanley Rd., St. Louis, Mo., 63144, USA

2. UNITY Available from Tripos Inc., 1699 South Hanley Rd., St. Louis, Mo., 63144, USA

3. FlexX Available from Tripos Inc., 1699 South Hanley Rd., St. Louis, Mo., 63144, USA

4. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

5. MCSS (Miranker, A. and M. Karplus, “Functionality Maps of Binding Sites: A Multiple Copy Simultaneous Search Method.” Proteins: Structure. Function and Genetics, 11, pp. 29-34 (1991)). MCSS is available from Molecular Simulations, Burlington, Mass.

6. AUTODOCK (Goodsell, D. S. and A. J. Olsen, “Automated Docking of Substrates to Proteins by Simulated Annealing”, Proteins: Structure. Function, and Genetics, 8, pp. 195-202 (1990)). AUTODOCK is available from Scripps Research Institute, La Jolla, Calif.

7. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once suitable compounds or chemical moieties have been selected, they can be assembled into a single compound or inhibitor. Assembly may be proceed by visual inspection of the relationship of the compounds or moieties to each other on the three-dimensional image displayed on a computer screen in relation to the structure coordinates of Pin1, Pin1 R14A and/or Pin1 R14A-inhibitor co-complexes. This could then be followed by manual model building using software such as Quanta or SYBYL.

Other useful programs to aid one of skill in the art in connecting the individual compounds or chemical entities include:

1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).

3. HOOK (available from Molecular Simulations, Burlington, Mass.).

In other embodiments, Pin1 inhibitors can be designed as a whole or “de novo” using either an empty active site or optionally including some portion(s) of a known inhibitor(s), e.g., PIN-051 and/or PIN-077, as described herein. Programs which can aid in these methods include:

1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.

2. LEGEND (Nishibata, Y. and A. Itai, Tetrahedron, 47, p. 8985 (1991)). LEGEND is available from Molecular Simulations, Burlington, Mass.

3. LeapFrog (available from Tripos Associates, St. Louis, Mo.).

Other molecular modeling techniques may also be employed in accordance with this invention. See, e.g., Cohen, N. C. et al., “Molecular Modeling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Once a compound has been designed or selected by the above methods, the efficiency with which that compound inhibits Pin1 can be tested and optimized by computational evaluation. For example, a compound that has been designed or selected to function as an Pin 1-inhibitor must also preferably traverse a volume not overlapping that occupied by the active site when it is bound to the native substrate. An effective Pin1 inhibitor must preferably demonstrate a relatively small difference in energy between its bound and free states (i.e., a small deformation energy of binding).

A compound designed or selected as an inhibitor of Pin1 can be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the inhibitor and the enzyme when the inhibitor is bound to Pin1 preferably make a neutral or favorable contribution to the enthalpy of binding.

Specific computer software is available in the art to evaluate compound deformation energy and electrostatic interaction. Examples of programs designed for such uses include: Gaussian 92, revision C, M. J. Frisch, Gaussian, Inc., Pittsburgh, Pa.; AMBER, version 4.0, P. A. Koliman, University of California at San Francisco; QUANTA/CHARMM, Molecular Simulations, Inc., Burlington, Mass.; and Insight II/Discover (Biosysm Technologies Inc., San Diego, Calif.). These programs may be implemented, for instance, using a Silicon Graphics workstation, IRIS 4D/35 or IBM RISC/6000 workstation model 550. Other hardware systems and software packages will be known to those skilled in the art.

Once a Pin1 inhibitor has been optimally selected or designed, as described herein, substitutions can then be made in some of its atoms or side groups in order to improve or modify its binding properties, again using the information provided by the interaction and specificity templates to identify regions amiable to modification. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to Pin1 by the same computer methods described in detail, above.

The interaction and/or specificity template also can be used to screen for and/or chemically optimize small molecules or chemical entities for Pin1 modulating activity, e.g., inhibiting activity. These molecules can be molecules previously identified as having Pin1 activity using a biological assay and/or can be molecules suspected of having such activity based on their structure. Examples of small molecules having one or more of the A-B-C-D-E moieties are described in copending applications PTZ-034, entitled “Pin1-Modulating Compounds and Methods of Use Thereof”, filed Mar. 3, 2003; PTZ-035, entitled “Pin1-Modulating Compounds and Methods of Use Thereof”, filed March 3, 2003; PTZ-036, entitled “Pin1-Modulating Compounds and Methods of Use Thereof”, filed Mar. 3, 2003; U.S. Provisional Application Serial No. 60/361,246, filed Mar. 1, 2002; U.S. Provisional Application Serial No. 60/361,206, filed Mar. 1, 2002; and U.S. Provisional Application Serial No. 60/361,231, filed Mar. 1, 2002 the entire contents of which are expressly incorporated by reference. Compounds described in the copending application were identified as Pin1 modulators using biological assays and further studied using the interaction template.

In certain embodiments of the invention, a modulator of Pin1 activity is administered in combination with other agents, or in conjunction with another, complementary treatment regime. For example, in one embodiment, a modulator of Pin1 activity is used to treat a cellular proliferation, growth, differentiation, and/or migration disorder. Accordingly, modulation of Pin1 activity may be used in conjunction with, for example, another agent or treatment used to treat the disorder, e.g., radiation or conventional chemotherapy.

The pharmaceutical compositions can be included in a container, pack, or dispenser together with instructions for administration.

The invention is further illustrated by the following examples, which should not be construed as further limiting. The contents of all references, pending patent applications and published patents, cited throughout this application are hereby expressly incorporated by reference. The animal models used throughout the Examples are accepted animal models and the demonstration of efficacy in these animal models is predictive of efficacy in humans.

### EXAMPLES

### Example 1

### Tissues with Elevated Pin1 Levels

Automated cellular imaging system ( ACIS) was used to determine tissues with elevated Pin1 Levels. The data that is presented in Example 1 is from U.S. patent application Ser. No. 10/071,747, filed Feb. 8, 2002, the entire contents of which are incorporated by reference.

Micro-histoarray sections were scanned and images were captured using the automated cellular imaging system (ChromaVision Medical Systems, Inc., San Juan Capistrano, Calif.) which combines automated microscopy and computerized image processing to analyze of multiple tissues on a single slide. ACIS was used to analyze microarray tissue sections on glass slides stained using a diaminodenzidine chromagen (DAB) and hematoxylin counterstain. Positive staining (brown color) as viewed by light microscope indicates the presence of the protein, and color intensity correlates directly with protein quantity (expression). The ACIS was able to recognize 255 levels of immnohistochemical staining intensity (0-255) and converted these to fractional scores for the selected individual areas. However, the base limit on the threshold for the Generic DAB is pre-set at 50 by the manufacturer because the system is very sensitive. Therefore, any intensity below 50 was treated as 0 in this study. Entire immunostained tissue sections were scanned using the 4×objective and images were captured using the 10×objective.

Calculation of Pin Protein Expression in Human Cancers

In this study, intensity scoring and the percent positive scoring (brown area was divided by total area) were used with the entire individual tissue dot selected. The immunohistochemical staining was quantitated without knowledge of a pathologist's score. All tissue samples were immunostained twice in University of Basel and in Pintex Pharmaceuticals, Inc. and the two data sets were evaluated in Pintex Pharmaceuticals, Inc. The final score was obtained by using the average of two data sets and calculated by the formulation:

score=intensity+(X percent positive staining).

The % of total cases showing elevated levels (over-expression) of Pin 1=[numbers of tumor samples with score larger than the score of the highest normal case)

total number of tumor samples

Results

TABLE 1
Pin1 protein over-expression in human tissues microarray

| | | % of Tumor Cases with |
|---|---|---|
| Tumor type | Case number | Eleveted Level of Pin1 |
| Brain tumor (3) | 111 | |
| Oligodendroglioma | 20 | 90 |
| Astrocytoma | 46 | 63 |
| Glioblastomamultiforme | 45 | 87 |
| Genecological tumor (13) | 372 | |
| Cervical carcinoma | 42 | 81 |
| Endometrium, endometroid | 46 | 0 |
| carcinoma | | |
| Endometrium, serous | 13 | 0 |
| carcinoma | | |
| Ovary, endometroid cancer | 45 | 24 |
| Ovary, Brenner tumor | 8 | 63 |
| Ovary mucinous cancer | 12 | 58 |
| Ovary, serous cancer | 47 | 43 |
| Uterus, carcinosarcoma | 6 | 100 |
| Breast, lobular cancer | 36 | 56 |
| Breast, ductal cancer | 47 | 47 |
| Breast, medullary cancer | 24 | 29 |
| Breast, mucinous cancer | 24 | 29 |
| Breast tubular cancer | 22 | 9 |
| Endocrine tumor (8) | 213 | |
| Thyroid adenocarcinoma | 42 | 29 |
| Thyroid follicular cancer | 49 | 41 |
| Thyroid medullary cancer | 8 | 100 |
| Thyroid papillary car | 36 | 22 |
| Parathyroid, adenocarcinoma | 28 | 21 |
| Adrenal gland adenoma | 15 | 0 |
| Adrenal gland cancer | 6 | 33 |
| Pheochromocytoma | 29 | 0 |
| Digestive tract tumor (11) | 411 | |
| Colon adenoma mild | 47 | 21 |
| displasia | | |
| Colon adenoma moderate | 47 | 17 |
| displasia | | |
| Colon adenoma severe | 49 | 14 |
| displasia | | |
| Colon adenocarcinoma | 43 | 2 |
| Esophagus adenocarcinoma | 43 | 30 |
| Hepatocelluar carcinoma | 34 | 62 |
| Mouth cancer | 46 | 93 |
| Gall bladder adenocarcinoma | 28 | 14 |
| Pancreatic adenocarcinoma | 43 | 2 |
| Small intestine | 10 | 0 |
| adenocarcinoma | | |
| Stomach diffuse | 21 | 0 |
| adenocarcinoma | | |
| Genitourinary tract tumor (9) | 381 | |
| Prostate (hormone-refract) | 44 | 59 |
| Prostate (untreated) | 47 | 64 |
| Kidney chromophobic | 15 | 0 |
| carcinoma | | |
| Kidney clear cell carcinoma | 47 | 0 |
| Kidney oncocytoma | 8 | 0 |
| Kidney papillary carcinoma | 44 | 0 |
| Testis, non-seminomatous | 43 | 2 |
| cancer | | |
| Testis seminoma | 47 | 2 |
| Urinary bladder transitional | 86 | 2 |
| carcinoma | | |
| Respiratory tract tumor (4) | 184 | |
| Lung, adenocarcinoma | 44 | 27 |
| Lung, large cell cancer | 45 | 42 |
| Lung, small cell cancer | 47 | 57 |
| Lung, squmous cell | 48 | 44 |
| carcinoma | | |
| Hematological neoplasia (5) | 146 | |
| Hodgkin lymphoma | 23 | 0 |
| MALT lymphoma | 47 | 4 |
| NHL, diffuse large B | 22 | 18 |
| NHL, others | 30 | 23 |
| Thymoma | 24 | 8 |
| Skin tumor (5) | 178 | |
| Skin, malignant melanoma | 44 | 73 |
| Skin, basolioma | 44 | 39 |
| Skin, squamous cell | 39 | 13 |
| cancer | | |
| Skin, merkel zell cancer | 5 | 100 |
| Skin benign nevus | 46 | 52 |
| Soft tissue tumor (2) | 45 | |
| Lipoma | 25 | 20 |
| Liposarcoma | 20 | 75 |

### Example 2:

### Effect of Pin1 Inhibitors on DU-145 Prostate Tumor Bearing Scid Mice

The effects of Pin1 inhibitors on tumor growth in the scid mouse human prostate tumor model are studied, first by screening scid mice for immunoglobulin (Ig) production by ELISA. The mice are then inoculated with the appropriate cancer cell line for the particular cancer being examined, (e.g., DU-145 prostate cancer cell line) using a subcutaneous flank injection in sterile saline on Day 0.

On approximately Day 16, mice with established tumors (˜40 mm³) are selected and divided into four groups. The first group receives a dosage of the vehicle control (e.g., DMSO, saline, water) on the following days, e.g., Days 16, 17, 18, 19, and 20. The second and third groups receives dosages of the Pin1 inhibitor. The fourth group, a positive control, receives a known pharmaceutical composition used to treat the particular type of cancer being examined. Each of the dosages is administered via intraperitoneal injection.

On the following days, the tumors in the mice are monitored, e.g., measured twice weekly and the volume of the tumors is estimated according to the formula: {(width)²×length]/2. Mice are weighed before commencing the experiment and weekly thereafter to check for signs of toxicity.

Based on the results of these experiments the efficacy of Pin1 inhibitors are determined.

We claim:

1. A method of treating a Pin1 associated disorder in a subject comprising,
administering to a subject an effective amount of an inhibitor of Pin1 activity having any one or more of the following moieties:
A-B-C-D-E
wherein A is a hydrophobic pocket interacting moiety;
wherein B is a cysteine/serine valley interacting moiety;
wherein C is a phosphate pocket interacting moiety;
wherein D is a substrate-entry groove interacting moiety; and
wherein E is a lip region interacting moiety;
such that said Pin1 disorder is treated.

2. The method of claim 1, wherein the Pin1 inhibitor comprises two of said moieties.

3. The method of claim 1, wherein the Pin1 inhibitor comprises three of said moieties.

4. The method of claim 1, wherein the Pin1 inhibitor comprises four of said moieties.

5. The method of claim 1, wherein the Pin1 inhibitor comprises A, B, C, D and E.

6. The method of any one of claims 1-5, wherein at least two of said moieties are covalently linked.

7. The method of claim 6, wherein at least 3 of the covalent moieties are covalently linked.

8. The method of claim 6, wherein at least 4 of the covalent moieties are covalently linked.

9. The method of claim 6, wherein five of the covalent moieties are covalently linked.

10. The method of claim 1, wherein said subject is administered a second chemotherapeutic agent.

11. The method of claim 7, wherein said second chemotherapeutic agent is radiation.
